(19) United States
(12) Patent Application Publication
(10) Pub. No.: US 2011/0231101 A1
(43) Pub. Date: Sep. 22, 2011

(54) BODY MOVEMENT ANALYSIS METHOD AND APPARATUS

(76) Inventors: Niranjan Bidargaddi, South Australia (AU); Antti Sarela, Queensland (AU); Mohan Karunanithi, Queensland (AU); Justin Boyle, Queensland (AU)

(21) Appl. No.: 12/673,929

(22) PCT Filed: Aug. 20, 2008
(86) PCT No.: PCT/AU2008/001221

§ 371 Date: Jun. 7, 2011

(30) Foreign Application Priority Data
Aug. 21, 2007 (AU) 2007904493

(51) Int. Cl. G06F 19/00; A61B 5/11

(52) U.S. Cl. 702/19

(57) ABSTRACT

A method and apparatus is disclosed for calculating a mobility performance index of a patient. The method includes the steps of: measuring an activity signal; identifying an activity type by segmenting said activity signal; calculating a measure for a selected said identified activity type; and calculating a mobility performance index from said measure.

### FIELD OF THE INVENTION

The present invention relates to signal processing and in particular to processing a body movement signal for determining body movements.

The invention has been developed primarily for use as a method and apparatus for monitoring movement of a person and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Existing methods used to determine a patient's walking speed and distance include GPS systems, radio localisation systems, camera systems, treadmills, and pedometer systems. However, GPS systems are typically limited to outdoor environments, radio localisation system operate substantially within a certain range of the radio transmitter/receiver, camera systems typically require line-of-sight visibility to a camera (often fixed to a particular location), treadmills reflect a set speed and are typically not indicative of the variable speed of walking during patients daily activities, and pedometer systems have potential errors with estimation of stride length and do not often collect time intervals associated with recorded step lengths.

A known technique for assessing the health status and physical condition of a patient is to evaluate cardiovascular and respiratory exercise performance by using the six-minute walking test. This test is widely accepted and used in clinical practice. The basic idea of the test is to measure the normal walking speed of the patient by measuring how far the patient travelled (or alternatively the speed) in six minutes.

The primary measurement observed in a six-minute test is the distance covered. The distance covered in a six-minute walk test is influenced by the patient's conditions such as cardiovascular diseases, chronic obstructive pulmonary diseases, neuromuscular disorders, etc. However, it would be appreciated that this test is carried out in a controlled environment (i.e., hospital). Such controlled tests potentially fail to highlight trends or functional capacity to walk in a free environment over a longer period of time. There is a need in the art for an alternative approach to measure the same functional capability in a free environment over a substantially continuous period of time.

There are also significant practical problems related to the six-minute test. The patient usually has to visit a care centre, and the test has to be performed under supervision. Because of these requirements, the test is typically not repeated often. Consequently, information is provided intermittently and very infrequently. Also because the test is performed in laboratory conditions, it does not give information on the patient's true walking ability in an independent living environment.

Other known methods and systems for monitoring patient activities at home usually provide only classification information i.e. the patient is walking, lying or standing. For example Aminian (U.S. Pat. No. 7,141,026) teaches a system and method for identifying the occurrence and duration of postural transitions, and Karason (U.S. Pat. No. 7,107,180) teaches a system and method of determining an activity level by ascertaining variables according to an activity type and calculating a formulaic activity index. These known devices typically measure the occurrence and duration of particular activities.

### OBJECT OF THE INVENTION

There is a need in the art for a method and system for accurately and reliably calculating speed and distance that a patient walks during a day.

It is an object of the invention in its preferred form to provide method and apparatus for body movement analysis.

According to a first aspect of the invention there is provided a method for calculating a mobility performance index of a patient, the method including the steps of
a) measuring an activity signal;
b) identifying an activity type by segmenting the activity signal;
c) calculating a measure for a selected identified activity type; and
d) calculating a mobility performance index from the measure.

Preferably the activity signal is indicative of acceleration.

The measure is preferably calculated using time-frequency analysis. Preferably, the measure is calculated using wavelet analysis. More preferably, the measure is calculated using a correlations function of the wavelet coefficients.

The mobility performance index is preferably indicative of the measure over a period of time. The mobility performance index is preferably indicative of a distribution of the measure over a period of time for displaying as a graph. Preferably, the mobility performance index includes statistical values indicative of the measure.

Preferably, the identified activity is walking, and wherein the measure is walking speed and distance walked. Alternatively, the identified activity is running, and wherein the measure is running speed and distance run.

Walking speed is preferably calculated using a correlations function of the wavelet coefficients. The distance walked is preferably calculated as the integral of walking speed over time. The mobility performance index is preferably any one or more of the indexes selected from the group including:
maximum distance travelled in a six minute interval during a day;
mean distance of all six minute intervals during a day; and
linear or non linear function of speed distributions of all six minute walk intervals in a day or week.

Preferably, the mobility performance index is indicative of a result of a six-minute walk test for a patient. The mobility performance index is preferably indicative of a distribution of the distance walked over 6 minute walking periods. The mobility performance index is preferably indicative of a distribution of the walking speed over 6 minute walking periods. The mobility performance index preferably includes statistical values indicative of variance and mean of the distributions.

Preferably, the mobility performance index is calculated intermittently. Alternatively, mobility performance index is calculated substantially continuously. The index may be calculated over relatively short or long time intervals.

The mobility performance index is preferably displayed graphically or numerically for assessing and observing the performance trends of a patient.

According to a first aspect of the invention there is provided an apparatus for calculating a mobility performance index, the apparatus comprising:
one or more of accelerometers for measuring an activity signal indicative of acceleration;
a processor adapted to:
a) identify an activity type by segmenting the activity signal;
b) calculate a measure for a selected identified activity type; and
c) calculate a mobility performance index from the measure.

The accelerometers preferably measure acceleration in at least two dimensions. More preferably, a accelerometers measure acceleration in three dimensions.

The measure is preferably calculated using time-frequency analysis. More preferably, the measure is calculated using wavelet analysis.

The activity signal is preferably indicative of acceleration and the processor identifies instances of an activity type of walking to calculate a measure of walking speed and distance walked.

The processor is preferably adapted to calculate mobility performance index substantially continuously. This mobility performance index is preferably any one or more of the indexes selected from the group including:
maximum distance travelled in a six minute interval during a day;
mean distance of all six minute intervals during a day; and
linear or non linear function of speed distributions of all six minute walk intervals in a day or week.

### BRIEF DESCRIPTION OF THE DRAWINGS

A preferred embodiment of the invention will now be described, by way of example only, with reference to the accompanying drawings in which:

FIG. 1 shows a flow chart for an example method for calculating a mobility performance index according to the invention;

FIG. 2 shows a block diagram of an example system for calculating a mobility performance index according to the invention;

FIG. 3 shows a patient wearing the measurement device of FIG. 2 on their waist;

FIG. 4 shows an activity signal indicative of a raw vertical acceleration signal for walking with varying speeds;

FIG. 5 shows intensities of scales obtained from wavelet decomposition of the activity signal of FIG. 4;

FIG. 6 shows the speeds calculation obtained by processing the signal of FIG. 5;

FIG. 7 shows example speed distributions derived over various periods of time;

FIG. 8 shows a functional measure calculated from the speed and distance distributions; and

FIG. 9 shows an example flow chart for processing an activity signal for calculating walking speed and walking distance.

### PREFERRED EMBODIMENT OF THE INVENTION

The health care of a patient can involve activity monitoring that requires the identifying and measuring of instances when a patient is walking, and then calculating a relatively accurate measure of the speed and distance travelled. This has been achieved in part though signal processing techniques, and in particular wavelet analysis of signals indicative of a patient's (or a person's) movement.

According to an embodiment, a method is disclosed that substantially provides the clinical information equivalent to a six-minute walk test to be to be calculated using measurements collected from the patient's own home environment without performing a specific supervised test in a care facility. This embodiment can also collect information continuously, which allows the clinicians to follow the true development of the patient's physical condition, or mobility. Because this enables data to be captured for substantially all the walking that the patient does, a more reliable measure of the patient's physical condition (or mobility) can be calculated than the measure provided by a six-minute walk test conducted in isolation.

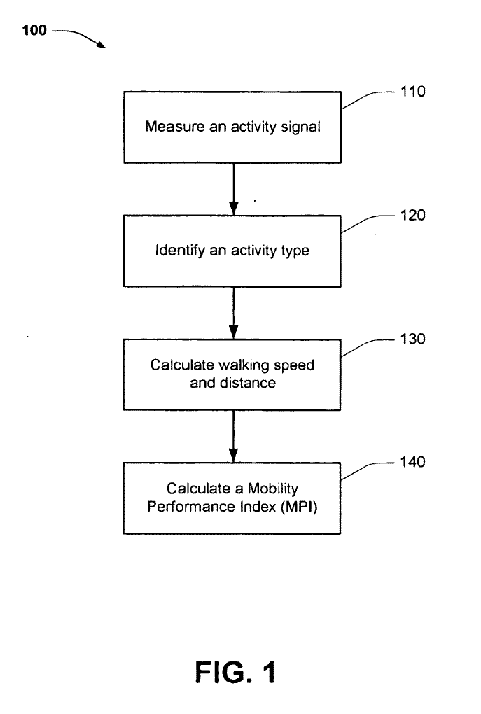

Referring to FIG. 1, an example embodiment of a method 100 for calculating a mobility performance index is disclosed. The method including the steps of:
a) measuring an activity signal 110;
b) identifying an activity type by segmenting the activity signal 120;
c) calculating a measure for a selected identified activity type 130; and
d) calculating a mobility performance index from the measure 140.

In an embodiment, the activity signal is indicative of acceleration, typically provided by a solid-state accelerometer. By processing this activity signal a period of walking can be identified, and the associated walking speed and walking distance calculated. Wavelet analysis is typically used in calculating walking speed and walking distance, and in identifying different walking patterns from the activity signal. It would be appreciated that this procedure is also suitable for other activities having periodic movement such as running. It would also be appreciated that other time-frequency analysis would be suitable for analysing particular activities.

A mobility performance index can be calculated based on these calculations of walking speed, walking distance and walking patterns, which are derived from raw measurements. This index can include simple measurements such as the distance travelled and maximum speed, and more sophisticated results such as distributions of the measurements over a period of time and statistical values associated with the measurements.

By way of example only, the mobility performance index can include:
a) maximum distance travelled in a six minute interval during a day;
b) mean distance of all six minute intervals during a day; and
c) linear or non-linear functions of speed distributions of all six minute walk intervals in a day or week.

For example, a mobility performance index can provide a measure indicative of a six-minute walk test result for a patient. The distribution of the walking speed (and/or distance walked) over 6 minute walking periods can also be included in the mobility performance index. Time-Frequency analysis, for example Wavelet analysis, can also be used to calculate an estimate for the energy expenditure from the activity signal.

In an embodiment the mobility performance index is calculated substantially continuously. In other embodiments the mobility performance index is calculated intermittently. In each case the index may be calculated over relatively short or long time intervals. Typically the resultant mobility performance index can be displayed graphically or numerically for assessing and observing the performance trends of a patient.

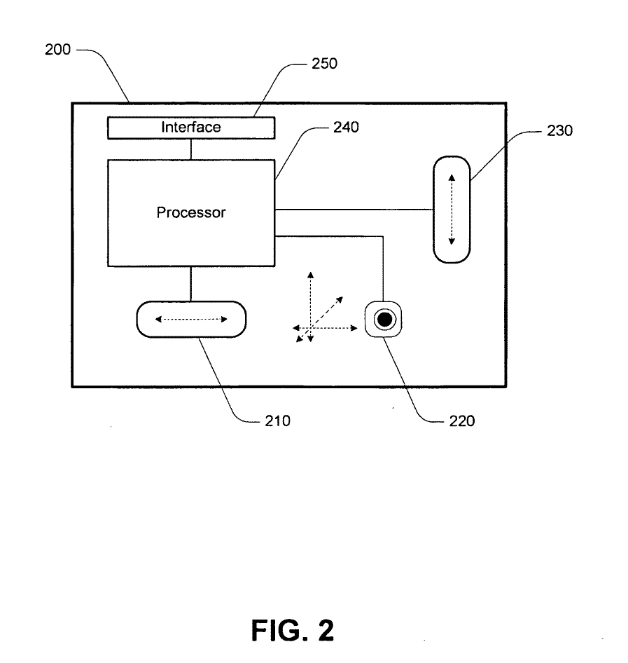

FIG. 2 shows an example embodiment of a measurement device 200, which can be used for calculating a mobility performance index. This device includes a plurality of accelerometers (210, 220, and 230) for measuring an activity signal indicative of acceleration. This measurement is typically provided by a solid-state accelerometer. The accelerometers are arranged such that acceleration can be measured in three dimensions. In this example, an accelerometer 210 measures horizontal sideward acceleration in one axis, an accelerometer 220 measures horizontal sideward acceleration in an axis perpendicular to the axis defined by the accelerometer 210, and an accelerometer 230 measures vertical acceleration. These signals are processed by a processor 240, and can be transferred to another processor via an interface 250. It is preferred that the interface is wireless, but it would be appreciated other wired interfaces can be used. Suitable accelerometer measurement devices use a variety of techniques to measure acceleration. These techniques include, but are not limited to, Piezo-film or piezoelectric sensor, Shear Mode Accelerometers, Surface Micromachined Capacitive (MEMS), Thermal (submicrometre CMOS process), Bulk Micromachined Capacitive, Bulk Micromachined Piezo Resistive, Capacitive Spring Mass Based, Electromechanical Servo (Servo Force Balance), Null-balance, Strain gauge, Resonance, Magnetic induction, Optical, Surface Acoustic Wave (SAW), Laser accelerometers, DC Response, High Temperature, Modally Tuned Impact Hammers, and Seat Pad Accelerometers.

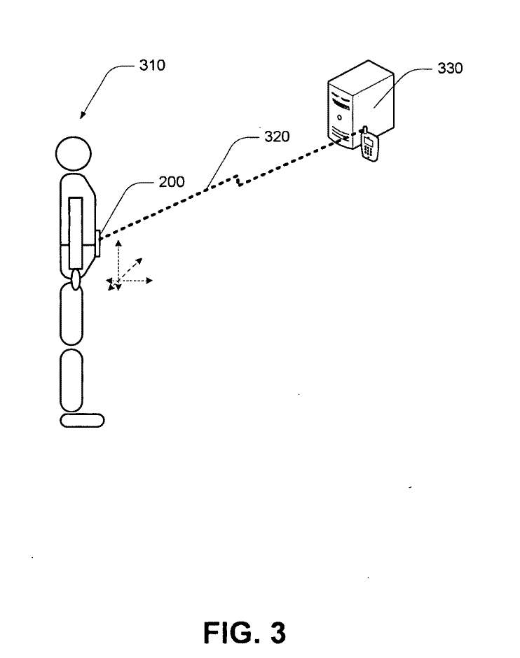

Referring to FIG. 3, an example embodiment is shown, where a patient 310 undergoing cardiac rehabilitation can wear a measurement device 200 having a three dimensional (3D) accelerometer unit on their waist. This device transmits, via a wireless link 320, the activity signal to a home hub 330. The hub further provides the raw measurement data to backend software that includes an algorithm module and user interface module for extracting and calculating the walking speeds and distance walked from the received data. These results can then be displayed. Clinicians can access this information and display the results for assessing the patient's mobility.

It would be appreciated that modifications and variations may be applied to these embodiments. By way of example only, the algorithms and user interface can be embedded in the measurement device or home hub depending on the application. Alternatively, the algorithms and user interface can be separated such that the algorithm is provided by the measurement device or hub, while the backend software provides the user interface.

It would be further appreciated that the measurement device signal, in some embodiments, can provide an activity signal indicative of 1-Dimensional (1D), 2-Dimensional (2D) or 3-Dimensional (3D) acceleration. An accelerometer or other sensor sensitive to body movements and can be used on waste, chest, wrist or upper arm etc. However, it is preferable to use a 3D accelerometer attached on waist.

Clinical information can be classified and calculated from the measured activity signal. By way of example only, an activity signal is measured during different activities of daily living by a waist worn 3D accelerometer. A rule based classification algorithms classifies and isolates portions of the activity signal that is indicative of walking (or other daily activities) from the raw activity signal. Calculating and displaying measures from the classified signal that describe the development of physical condition of a patient are then performed.

The six-minute walk test is known by physicians for assessing a patient's functional capacity. The primary measurement observed in a six-minute test is the distance covered (or alternatively the speed). An aspect of the present invention provides an alternative approach to measure the same functional capability in a free environment over a substantially continuous period of time.

An embodiment is disclosed which performs speed and distance based walking performance assessment of a patient from measurements taken in the patients natural environment, using a single waist mounted accelerometer device, as an alternative to the six-minute walk test carried out in a clinical environment.

Wavelet analysis of an activity signal, in the form of accelerometer data can be used in assessing a patient's functional capacity. Accelerometers sense motion by detecting acceleration and deceleration in one or more directions of movement. When these devices detect movement, an electric current is generated within the sensor that is proportional to the acceleration of the device.

Accelerometers can also be used to measure step counts and for classification of motions and postures such as sitting, standing, walking, transitions and other miscellaneous activity.

Wavelet based analysis is used to provide a relatively high resolution time localization of the observed frequency components. This is advantageous given the typical non-stationary nature of biomedical signals.

Walking activity can be classified from an anterior-posterior and vertical acceleration signal using wavelet analysis. The frequencies of the anterior-posterior and vertical acceleration measured from an accelerometer device mounted on the trunk (centre of mass) typically range from 0.6 to 2.5 Hz for walking activity.

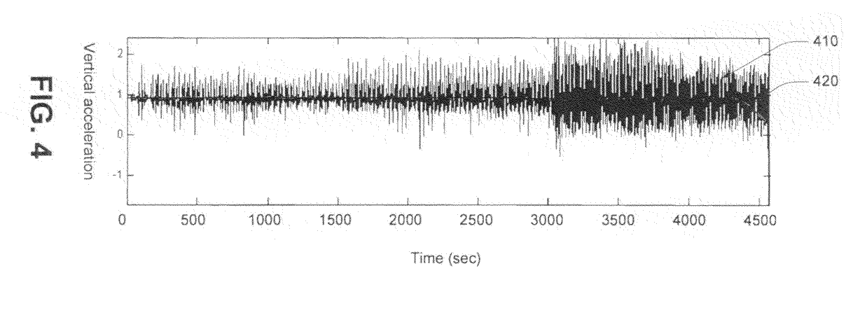

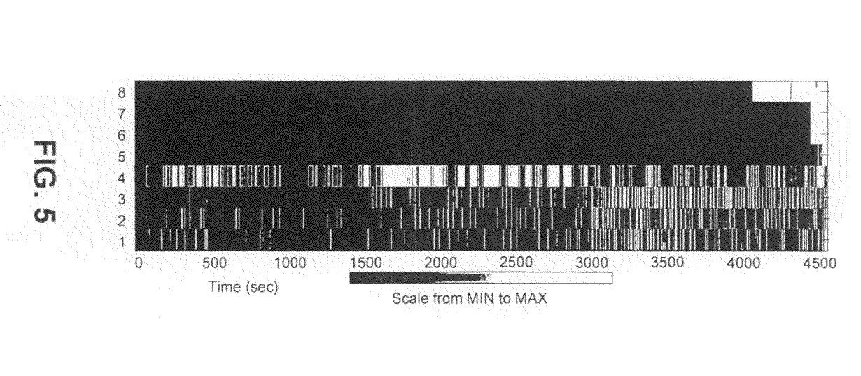

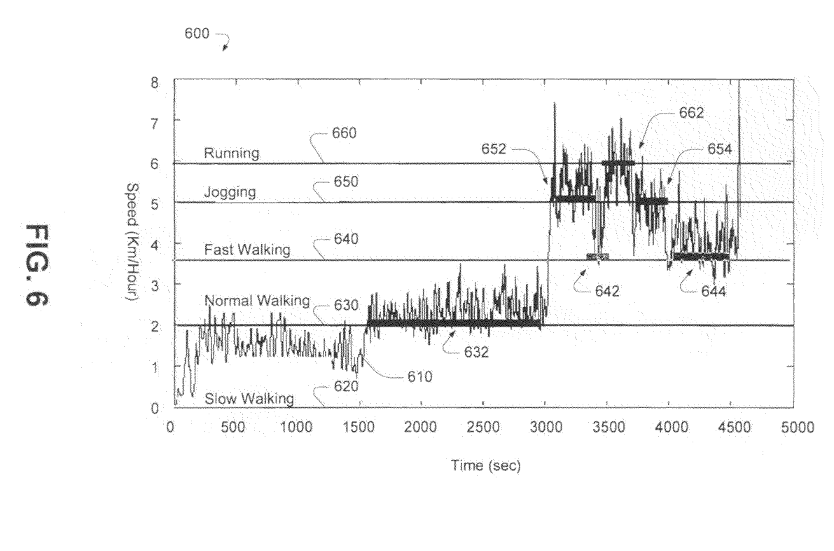

FIG. 4 though FIG. 6 are illustrative of the processing of an activity signal to derive a measure of walking speed. FIG. 4 shows a measured activity signal indicative of a raw vertical acceleration signal 410 and filtered vertical acceleration signal 420 for walking with varying speeds. The x-axis is time in seconds and the y-axis is indicative of acceleration in units of 1 g=9.8 m/sec². FIG. 5 shows intensities of scales obtained from wavelet decomposition for walking, i.e. the value of wavelet coefficient of scale i at time t for detail signal Xᵢ. The x-axis is time in seconds and the y-axis is the scale value and the intensity is indicative of the wavelet coefficients (intensity/power). FIG. 6 shows the speeds obtained by processing the activity signal. The x-axis is time in seconds and the y-axis is the speed in km/hour.

Referring to FIG. 6 shows, by way of example only, the speeds obtained by processing the activity signal. The x-axis is time in seconds and the y-axis is the speed in km/hour. The speed signal 610, is used to identify periods of “slow walking”, “normal walking”, “fast walking” “jogging” and “running” as identified by graphs 620, 630, 640, 650 and 660. In this example; a period of normal walking is indicated by the graph portion 632, periods of fast walking are indicated by the graph portions 642 and 644, periods of jogging are indicated by the graph portions 652 and 654, and a period of running is indicated by the graph portion 662.

Referring to FIG. 5, the raw vertical acceleration signal is decomposed into eight detailed signals at wavelet scales 1 to 8 by applying a discrete wavelet transform utilising a Daubechies ‘db10’ mother wavelet. It would be appreciated that Wavelet transformation involves decomposing a signal into its detail (high frequency) and approximation (low frequency) component signals. The Daubechies db10 wavelets provide two main oscillations that correspond to the minimum of two steps period in walking activity. It would be appreciated that other wavelet transforms can be applied for varying degrees of accuracy and reliability. In this example, wavelet scales 3 and 4 (X₃, X₄) are indicative of most walking activity. Such detail signals can be further used to distinguish between walking on stairs and level ground.

The applications of the discrete wavelet transform analysis of accelerometer signals to determine daily activity classification can be used to identify walking periods and classification of walking on level grounds and on stairways. The discrete wavelet transform analysis of accelerometer signals can be further applied to determining walking speed.

By calculating a measure of energy (or power) for detail signal Xᵢ, at wavelet scale i using its wavelet coefficients, it is possible to observe their variation with different speeds of walking at wavelet scales predominantly at levels 2, 3 and 4. By way of example, X₄ is typically more prominent with slow walking, while X₃ and X₂ are typically more prominent at higher speed of walking. The energy also varies with stride length, indicative of a correlation between the energy components to the speed of walking. As the calculated energy varies with respect to walking patterns and stride length, the speed measure obtained through this technique is typically more robust and accurate compared to other known techniques.

By way of example, speed S, can be calculated during different phases of walking at any instant of time t, as:

Sₜ=ƒ(I(X₁,t),I(X₂,t),I(X₃,t), . . . ,I(Xₘ,t)).

In the above equation I(Xᵢ,t) corresponds to the energy/power observed in details Xᵢ of wavelet scale i (1<i<m) at time t.

The speed Sₜ is provided by the correlation function ƒ(.). It would be appreciated that this function can be linear or quadratic. By way of example, Sₜ is provided by the function:

$$\begin{matrix}{S}_{t} = {a ( {{b}_{1} ( {x}_{1 , t} )}^{n} + {{b}_{2} ( {x}_{2 , t} )}^{n} + … , {{b}_{m} ( {x}_{m , t} )}^{n} )}^{\frac{1}{n}} \\ = {a ( {\sum }_{i = 1}^{m} {{b}_{i} ( {x}_{i , t} )}^{n} )}^{\frac{1}{n}} .\end{matrix}$$

In this equation I(Xᵢ,t)=x_{i,t} (the value of wavelet coefficient of scale i at time t), and bᵢ is the correlation constant. In the example, and as shown in FIGS. 4 through 6, m=8, n=2 and bᵢ=1 (1≦i≦m).

The wavelet transform of acceleration signal, up to scale m, provides m detail signals Xᵢ, . . . , Xₘ. At any given time t the energy/power for detail signal Xᵢ at wavelet scale, i.e. I(Xᵢ,t)=x_{i,t} is obtained by integrating Xᵢ with a mother wavelet db10 of scale i. This integration of Xᵢ over time provides a measure x_{i,t} indicative of the speed component for scale i.

The speed Sₜ is provided by the correlation function ƒ(.) (as shown above) to combine these different speed components to obtain a measure of speed.

These speed components can be combined in a quadratic way to obtain speed as shown above. Obtaining different speed components, at instances of time t, by integrating the signal with different scales inherently take into account the varying stride lengths present during walking activities.

It would be appreciated that the distance covered during walking can be obtained by integrating speed with walking duration as:

D=∫Sₜdt.

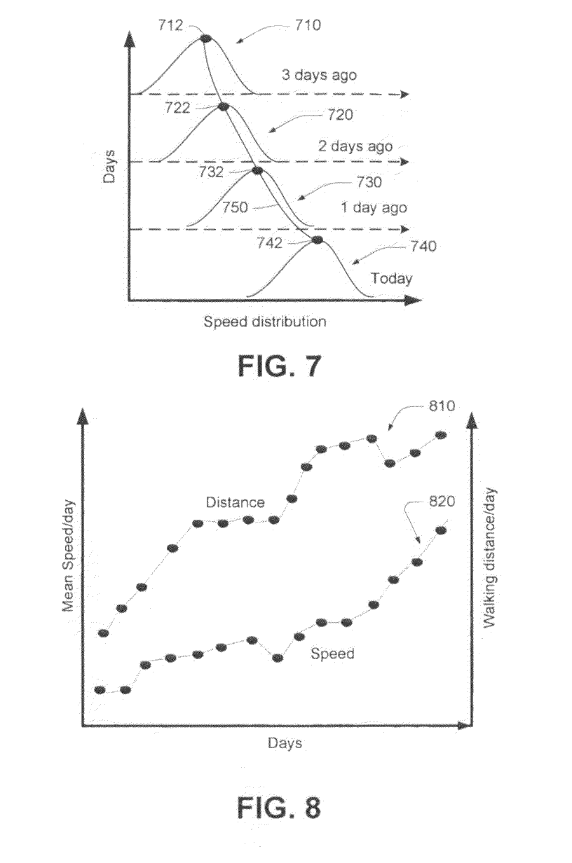

Referring to FIG. 7, by way of example only, a speed distributions of Sₜ can be calculated over a significant period (for example a day or week). From this speed distribution, a normalized speed pattern distribution can be obtained. By processing the variance of these speed distributions over the time period, it is possible to assess the walking ability of the patient. The distance covered can be used along with speed pattern distribution to enable physiotherapists to assess the mobility (or walking ability) of a patient undergoing rehabilitation assessment. In this example, daily distributions 710, 720, 730, and 740 are each calculated for a respective one of four days. A mean speed 712, 722, 732, and 742 can be identified for respective distributions 710, 720, 730, and 740.

A mobility performance index (MPI), being a functional measure, can then be calculated from the speed and distance distributions. Mobility performance index is a measure used to assess the progress of walking ability (or physical condition) of a patient in a daily living environment (both in hospital and home). This measure is preferably obtained daily by typically calculating the distance travelled in a six-minute walk interval. As there can be more than one six-minute walking interval within any given day, a MPI can be calculated in a number of ways. By way of example only, MPI can be calculated on the basis of
the maximum distance travelled for all the six-minute interval;
the mean distance of all six-minute intervals;
the mean walking speed for a day; and
a linear (or non-linear) function of the above.

Further weightings may be applied to account for other factors, for example the body mass index, age, medical history, prescription drugs and chronic disease types.

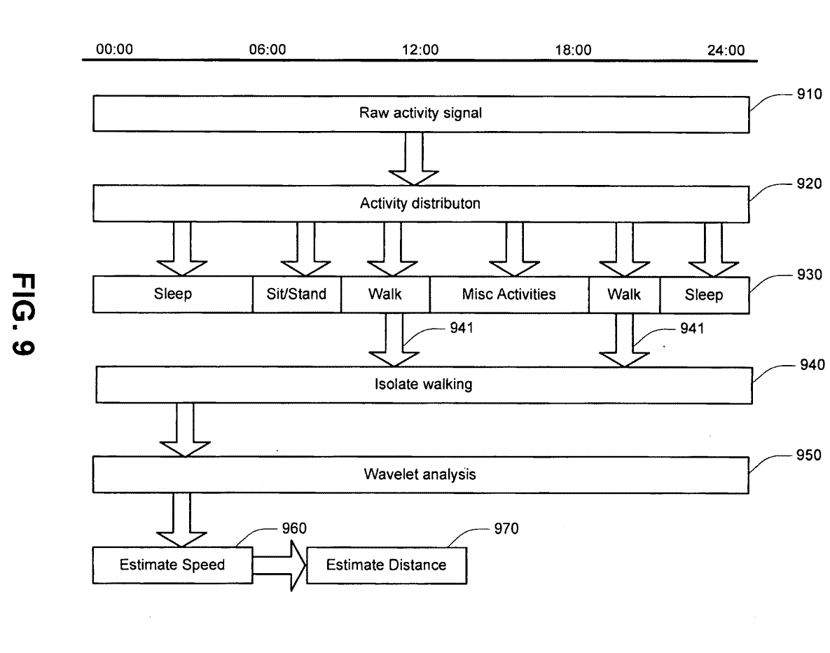

Referring to FIG. 8, the values of MPI can be plotted over each day to obtain an MPI curve. This curve shows the trends of the rehabilitation or mobility performance over a specified period of time. By way of example only, the walking distance per day 810, and the mean speed per day 820 are plotted.

It would be appreciated that this measure of MPI:
provides a continuous quantitative measure for assessment compared to just a visual assessment of a short period;
provides the trends of walking continuously over a long period of time;
is a quantitative assessment that is relatively accurate and consistent compared to visual assessment that is subject to human error (particularly between different assessors);
can be conducted in a free living environment compared to the controlled environment of a hospital; and
can be conducted in a natural environment without obstructing the subject's normal activities and time.

It would be appreciated that this analysis does not require leg length and step length to calculate speed or distance covered and is therefore suitable for a wide range of patients.

The consecutive six-minute walking test speed distribution graphs can be presented in a single view so that it is possible to analyze the development and changes of the physical condition.

It is possible to further calculate, view and trend statistical values from the consecutive speed distributions e.g. mean, maximum, minimum or standard deviation, which provide additional information on the development of the person's walking activity and physical condition.

The walking speed and distance measure can be derived from a single device worn on the waist without the need for tethered cabling or sensors placed on multiple parts of the body. It would be appreciated that the disclosed embodiments provide improvements over known methods used to determine a patient's walking speed and distance, such as GPS systems radio localisation system, camera systems, treadmills, and pedometer systems.

Referring to FIG. 9, there is illustrated a data flows diagram of a process according to an embodiment. Initially, the raw activity signal 910 is classified by classifying 920 regions of the signal into a plurality of identifiable activities 930. A selected activity is isolated for further processing 940. In this example, instances of walking 941 are isolated and undergo wavelet analysis 950. From this wavelet analysis, the speed 960 and distance 970 are calculated.

The disclosed embodiment can provide increased information continuously, quicker, and cheaper than the intermittent and infrequent method that is currently in normal clinical use. This increased information content can provide the clinician better understanding of the development of person's physical condition. The described embodiments overcomes limitations of existing methods that can be used to quantify walking speed. These embodiments are not limited by:
outdoor environments such as Global Positioning Systems (GPS);
Range such as radio localization and tracking systems; and
Location such as treadmill and camera systems.

An embodiment can further eliminate inaccurate speed/distance measurement from pedometers based on user input of stride length.

It would be appreciated that an embodiment can have wide impact in the health care sector (including Physiotherapy, Rehabilitation and Geriatry) and Sports Medicine sector, and can be applied to other disease or care process that would benefit from follow up of the patient's physical condition.

By way of example only, an embodiment can provide any one or more of the following impacts to the health care sector:
streamlined duties for physiotherapists performing six minute walking test;
reduced travel and transportation costs for patient;
reduced avoidable patient visits to public and private hospitals, GPs, private practice specialists, outpatient physiotherapy session and associated medical expenses;
increasing physical activity levels in the community & all associated benefits by potentially better assessing falls risk;
increasing functional mobility after discharge;
identifying fitness trends;
improved communication of current health information, leading to improved continuum of care;
improved clinical pathways for chronic disease patients involving modified exercise programs leading to reduced lengths of stay and reduced readmissions;
faster service delivery (improved productivity); patient information arrives to clinician faster; can review many patients in the time it took previously for one;
fewer errors than when patient observations entered manually; and
more complete data information obtainable about patient movement in the home during normal daily activities.

It will be appreciated that the illustrated embodiments provide a system and method of identifying and measuring the instances in which a patient is walking and calculate a relatively accurate measure of the speed and distance travelled. An additional application can include patient localization and tracking, especially indoors where GPS is typically less effective. Localization could be improved by incorporating real-time estimates of the persons walking speed and travelled distance. This information combined with the walking direction measured with e.g. a magnetometer can be used to improve the location estimate.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not.

Unless specifically stated otherwise, as apparent from the following discussions, it is appreciated that throughout the specification discussions utilizing terms such as “processing”, “computing”, “calculating”, “determining” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

Although the invention has been described with reference to specific examples, it will be appreciated by those skilled in the art that the invention may be embodied in many other forms.

1. A method for calculating a mobility performance index of a patient, said method including the steps of:
(a) measuring an activity signal;
(b) identifying an identified activity type by segmenting said activity signal;
(c) calculating a measure for a selected said identified activity type; and
(d) calculating a mobility performance index from said measure.

2. The method according to claim 1 wherein said activity signal is indicative of acceleration.

3. The method according to claim 1 wherein said measure is calculated using time-frequency analysis.

4. The method according to claim 1 wherein said measure is calculated using wavelet analysis.

5. The method according to claim 4 wherein said measure is calculated using a correlations function of the wavelet coefficients.

6. The method according to claim 1 wherein said mobility performance index is indicative of said measure over a period of time.

7. The method according to claim 1 wherein said mobility performance index is indicative of a distribution of said measure over a period of time for displaying as a graph.

8. The method according to claim 1 wherein said mobility performance index includes statistical values indicative of said measure.

9. The method according to claim 1 wherein said identified activity is running, and wherein said measure is running speed and distance run.

10. The method according to claim 1 wherein said identified activity is walking, and wherein said measure is walking speed and distance walked.

11. The method according to claim 10 wherein said walking speed is calculated using a correlations function of the wavelet coefficients.

12. The method according to claim 10 wherein said distance walked is calculated as the integral of walking speed over time.

13. The method according to claim 10 wherein said mobility performance index is any one or more of the indexes selected from the group including:
(a) maximum distance travelled in a six minute interval during a day;
(b) mean distance of all six minute intervals during a day; and
(c) linear or non linear function of speed distributions of all six minute walk intervals in a day or week.

14. (canceled)

15. (canceled)

16. (canceled)

17. The method according to claim 10 wherein said mobility performance index is indicative of a result of a six-minute walk test for said patient.

18. The method according to claim 10 wherein said mobility performance index is indicative of a distribution of said distance walked over 6 minute walking periods.

19. The method according to claim 10 wherein said mobility performance index is indicative of a distribution of said walking speed over 6 minute walking periods.

20. The method according to claim 18 wherein said mobility performance index includes statistical values indicative of variance and mean of said distributions.

21. The method according to claim 1 wherein said mobility performance index is calculated substantially continuously.

22. The method according to claim 1 wherein said mobility performance index is calculated intermittently.

23. The method according to claim 1 wherein said mobility performance index is displayed graphically or numerically for assessing and observing the performance trends of a patient.

24. (canceled)

25. An apparatus for calculating a mobility performance index, said apparatus comprising:
one or more of accelerometers for measuring an activity signal indicative of acceleration;
a processor adapted to:
(a) identify an activity type by segmenting said activity signal;
(b) calculate a measure for a selected said identified activity type; and
(c) calculate a mobility performance index from said measure.

26. The apparatus according to claim 25 wherein said accelerometers measure acceleration in at least two dimensions.

27. The apparatus according to claim 26 wherein said accelerometers measure acceleration in three dimensions.

28. The apparatus according to claim 25 wherein said measure is calculated using time-frequency analysis.

29. The apparatus according to claim 25 wherein said measure is calculated using wavelet analysis.

30. The apparatus according to claim 25 wherein said activity signal is indicative of acceleration and said processor identifies instances of an activity type of walking to calculate a measure of walking speed and distance walked.

31. The apparatus according to claim 25 wherein said processor is adapted to calculate mobility performance index substantially continuously.

32. The apparatus according to claim 25 wherein said mobility performance index is any one or more of the indexes selected from the group including:
maximum distance travelled in a six minute interval during a day;
mean distance of all six minute intervals during a day; and
linear or non linear function of speed distributions of all six minute walk intervals in a day or week.

33. (canceled)
